SEQUENCE LISTING (2 sequences)

SEQ ID NO: 1 - DNA, 21 bp, Artificial sequence
  Synthetic
cactggactaaacttcagcat

SEQ ID NO: 2 - DNA, 21 bp, Artificial sequence
  Synthetic
gcatagcgtaatgtccatgtt

(19) United States
(12) Patent Application Publication
(10) Pub. No.: US 2024/0271142 A1
(43) Pub. Date: Aug. 15, 2024

(54) COMPOSITIONS AND METHODS FOR THE INHIBITION OF NERVE GROWTH FACTOR AND THE TREATMENT/PREVENTION OF ATRIAL FIBRILLATION

(71) Applicant: NORTHWESTERN UNIVERSITY, Evanston, IL (US)

(72) Inventor: Rishi ARORA, Evanston, IL (US)

(21) Appl. No.: 18/570,532

(22) PCT Filed: Jun. 14, 2022
(86) PCT No.: PCT/US2022/033444

§ 371 Date: Dec. 14, 2023

Related U.S. Application Data
(60) Provisional application No. 63/210,338, filed on Jun. 14, 2021.
(60) Provisional application No. 63/237,933, filed on Aug. 27, 2021.

(51) Int. Cl. C12N 15/113; A61B 5/00; A61B 5/367; A61K 9/00; A61P 9/06

(52) U.S. Cl. CPC C12N 15/1136; A61B 5/367; A61B 5/4848; A61K 9/0019; A61P 9/06; C12N 2310/14; C12N 2310/531

(57) ABSTRACT

Provided herein are compositions and methods for the inhibition of nerve growth factor (NGF) and the treatment/prevention of atrial fibrillation. In particular, inhibitors of NGF expression are administered to the myocardial tissue of a subject to treat or prevent atrial fibrillation and/or autonomic nerve sprouting in the atria.

### CROSS-REFERENCE TO RELATED APPLICATIONS

This application claims the benefit of U.S. Provisional Patent Application No. 63/210,338, filed on Jun. 14, 2021, and U.S. Provisional Patent Application No. 63/237,933, filed on Aug. 27, 2021, both of which are incorporated by reference herein.

### STATEMENT REGARDING FEDERAL FUNDING

This invention was made with government support under HL 140061 awarded by the National Institutes of Health. The government has certain rights in this invention.

### SEQUENCE LISTING

The text of the computer readable sequence listing filed herewith, titled “39552-601_SEQUENCE_LISTING_ST25”, created Jun. 14, 2022, having a file size of 652 bytes, is hereby incorporated by reference in its entirety.

### FIELD

Provided herein are compositions and methods for the inhibition of nerve growth factor (NGF) and the treatment/prevention of atrial fibrillation. In particular, inhibitors of NGF expression are administered to the myocardial tissue of a subject to treat or prevent atrial fibrillation and/or autonomic nerve sprouting in the atria.

### BACKGROUND

Atrial Fibrillation (AF) is the most common heart rhythm disorder (Benjamin E J, Levy D, Vaziri S M, D'Agostino R B, Belanger A J, Wolf P A. “Independent risk factors for atrial fibrillation in a population-based cohort. The Framingham Heart Study,” JAMA 1994; 271:840-4; incorporated by reference in its entirety), and is a major risk factor for stroke and HF (Balasubramaniam R, Kistler P M. AF and “Heart failure: the chicken or the egg?” Heart 2009; 95:535-9; Lakshminarayan K, Anderson D C, Herzog C A, Qureshi A I. “Clinical epidemiology of atrial fibrillation and related cerebrovascular events in the United States,” Neurologist 2008; 14:143-50; Lip G Y, Kakar P, Watson T. “Atrial fibrillation—the growing epidemic” [comment], Heart 2007; 93:542-3; incorporated by reference in their entireties). Current strategies for addressing AF, such as electroablation, do not address the specific mechanisms underlying AF (Ben Morrison T, Jared Bunch T, Gersh B J. “Pathophysiology of concomitant atrial fibrillation and heart failure: implications for management,” Nat. Clin. Pract. Cardiovasc. Med 2009; 6:46-56; incorporated by reference in its entirety). Recent research has therefore attempted to better define the mechanisms underlying AF, in order to improve upon the success of ablation and to develop new biological therapies for AF.

### SUMMARY

Provided herein are compositions and methods for the inhibition of nerve growth factor (NGF) and the treatment/prevention of atrial fibrillation. In particular, inhibitors of NGF expression are administered to the myocardial tissue of a subject to treat or prevent atrial fibrillation and/or autonomic nerve sprouting in the atria.

In some embodiments, provided herein are methods of treating and/or preventing atrial fibrillation (AF) in a subject, comprising administering an effective amount of a nerve growth factor (NGF) inhibitory agent to the subject. In some embodiments, the subject suffers from AF. In some embodiments, the subject is at elevated risk of AF. In some embodiments, the NGF inhibitory agent inhibits the expression of NGF. In some embodiments, the NGF inhibitory agent comprises a nucleic acid. In some embodiments, administering the nucleic acid comprises administering a vector (e.g., plasmid, viral vector, non-viral vector, etc.) and/or transgene encoding the nucleic acid and allowing the nucleic acid to be expressed within the cells of the subject. In some embodiments, administering the nucleic acid comprises directly administering the nucleic acid to the subject. In some embodiments, the NGF inhibitory agent is administered to the myocardial tissue of the subject. In some embodiments, the myocardial tissue comprises atrial or ventricle tissue. In some embodiments, the NGF inhibitory agent is administered to the left atrial appendage. In some embodiments, the nucleic acid is an antisense RNA, short hairpin RNA (shRNA), short interfering RNA (siRNA), or microRNA (miRNA). In some embodiments, the nucleic acid is an NGF shRNA comprising at least 70% (e.g., 70%, 75%, 80%, 85%, 90%, 95%, 100%, or ranges therebetween) sequence identity with SEQ ID NO: 1. In some embodiments, administering the NGF inhibitory agent comprises injecting the NGF inhibitory agent into the tissue of the subject. In some embodiments, injecting is by needleless injection. In some embodiments, injecting is by microneedle injection. In some embodiments, methods further comprise assessing a parameter of myocardial tissue (e.g., atrial tissue) status in the subject. In some embodiments, assessing a parameter of atrial tissue status in the subject comprises monitoring an electrophysiological measurement associated with AF or assessing nerve sprouting for a region of the myocardial tissue before and/or after administering the NGF inhibitory agent to the subject. In some embodiments, assessing a parameter of atrial tissue status in the subject comprises monitoring an electrophysiological measurement associated with AF selected from AF onset, AF duration, AF episode inducibility, effective refractory periods, conductivity, and conductive inhomogeneity index.

In some embodiments, provided herein are compositions (e.g., pharmaceutical compositions) comprising a nucleic acid capable of inhibiting expression of nerve growth factor (NGF). In some embodiments, the nucleic acid is an antisense RNA, short hairpin RNA (shRNA), short interfering RNA (siRNA), or microRNA (miRNA). In some embodiments, the nucleic acid is a vector (e.g., plasmid, viral vector, non-viral vector, etc.) or transgene encoding an antisense RNA, short hairpin RNA (shRNA), short interfering RNA (siRNA), or microRNA (miRNA). In some embodiments, the nucleic acid is an isolated nucleic acid encoding a small hairpin RNA against NGF mRNA. In some embodiments, the NGF shRNA comprises at least 70% (e.g., 70%, 75%, 80%, 85%, 90%, 95%, 100%, or ranges therebetween) sequence identity with SEQ ID NO: 1.

In some embodiments, provided herein is the use of a composition (e.g., pharmaceutical compositions) comprising an NGF inhibitory agent herein in the treatment or prevention of AF. In some embodiments, provided herein is the use of a composition (e.g., pharmaceutical compositions) comprising an NGF inhibitory agent herein as a medicament. In some embodiments, provided herein is the use of a composition (e.g., pharmaceutical compositions) comprising a an NGF inhibitory agent herein the manufacture of a medicament.

### BRIEF DESCRIPTION OF THE DRAWINGS

FIG. 1. Targeted injection of NGF shRNA in the left atrial appendage prevents RAP induced AF.

FIG. 2A-B. Targeted injection of NGF shRNA in the left and right atria prevents RAP induced AF over the timespan of (A) 28 days and (B) 12 weeks.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an inhibitory agent” is a reference to one or more inhibitory agents and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.” As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

As used herein, the term “preventing” refers to prophylactic steps taken to reduce the likelihood of a subject (e.g., an at-risk subject) from developing or suffering from a particular disease, disorder, or condition (e.g., AF). The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease/condition/symptom in a subject suffering from the disease/condition/symptom.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., an NGF inhibitor and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “pharmaceutically acceptable salt” refers to any pharmaceutically acceptable salt (e.g., acid or base) of a compound of the present invention which upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “instructions for administering said compound to a subject,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

As used herein, the term “operably-linked” refers to the association of nucleic acid sequences on a polynucleotide so that the function of one of the sequences is affected by another. For example, a regulatory DNA sequence is said to be “operably linked to” a DNA sequence that codes for an RNA (“an RNA coding sequence” or “shRNA encoding sequence”) or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. An RNA coding sequence refers to a nucleic acid that can serve as a template for synthesis of an RNA molecule such as an shRNA. Preferably, the RNA coding region is a DNA sequence.

As used herein, the term “promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that stimulates promoter activity and may be an innate element of the promoter or a heterologous element inserted to enhance the level or tissue specificity of a promoter. It is capable of operating in both orientations (sense or antisense), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Any promoter known in the art which regulates the expression of the shRNA or RNA coding sequence is envisioned in the practice of the invention.

As used herein, the term “reporter element” or “marker” is meant a polynucleotide that encodes a polypeptide capable of being detected in a screening assay. Examples of polypeptides encoded by reporter elements include, but are not limited to, lacZ, GFP, luciferase, and chloramphenicol acetyltransferase. See, for example, U.S. Pat. No. 7,416,849. Many reporter elements and marker genes are known in the art and envisioned for use in the inventions disclosed herein.

As used herein, the term “RNA transcript” refers to the product resulting from RNA polymerase catalyzed transcription of a DNA sequence. “Messenger RNA transcript (mRNA)” refers to the RNA that is without introns and that can be translated into protein by the cell.

As used herein, the term “shRNA” (small hairpin RNA) refers to an RNA duplex wherein a portion of the RNA is part of a hairpin structure (shRNA). In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some aspects, the overhang is a 3′ or a 5′ overhang 0, 1, 2, 3, 4 or 5 nucleotides in length. In one aspect of this invention, a nucleotide sequence in the vector serves as a template for the expression of a small hairpin RNA, comprising a sense region, a loop region and an antisense region. Following expression the sense and antisense regions form a duplex. It is this duplex, forming the shRNA, which hybridizes to, for example, the NGF mRNA and reduces expression of NGF, reducing nerve sprouting and/or, treating and/or preventing AF.

As used herein, the term “knock-down” or “knock-down technology” refers to a technique of gene silencing in which the expression of a target gene or gene of interest is reduced as compared to the gene expression prior to the introduction of the siRNA, which can lead to the inhibition of production of the target gene product. “Double knockdown” is the knockdown of two genes. The term “reduced” is used herein to indicate that the target gene expression is lowered by 0.1-100%. For example, the expression may be reduced 0.5, 1, 5, 10, 15, 20, 25, 30, 35, 40, 45, 50, 55, 60, 65, 70, 75, 80, 85, 90, 95, or even 99%. The expression may be reduced by any amount (%) within those intervals, such as for example, 2-4, 11-14, 16-19, 21-24, 26-29, 31-34, 36-39, 41-44, 46-49, 51-54, 56-59, 61-64, 66-69, 71-74, 76-79, 81-84, 86-89, 91-94, 96, 97, 98 or 99. Knock-down of gene expression can be directed by the use of shRNAs.

As used herein, the term “vector” refers to any viral or non-viral vector, as well as any plasmid, cosmid, phage or binary vector in double or single stranded linear or circular form that may or may not be self-transmissible or mobilizable, and that can transform prokaryotic or eukaryotic host cells either by integration into the cellular genome or which can exist extrachromosomally (e.g., autonomous replicating plasmid with an origin of replication). Any vector known in the art is envisioned for use in the practice of this invention.

### DETAILED DESCRIPTION

Provided herein are compositions and methods for the inhibition of nerve growth factor (NGF) and the treatment/prevention of atrial fibrillation. In particular, inhibitors of NGF expression are administered to the myocardial tissue of a subject to treat or prevent atrial fibrillation and/or autonomic nerve sprouting in the atria.

Embodiments herein provide compositions and methods for inhibiting NGF expression and/or activity in a subject suffering from atrial fibrillation. Certain embodiments comprise inhibiting the expression of NGF to reduce/inhibit/prevent autonomic nerve sprouting in the atria. Experiments were conducted during development of embodiments herein using an NGF inhibitor based upon RNA interference (RNAi) with small hairpin RNA directed against the NGF mRNA (NGF shRNA). Pharmaceutical compositions based upon NGF shRNA inhibit expression of the NGF gene, resulting in lower incidence of AF. The principle of NGF inhibition for the treatment of AF is readily extendable from the exemplary NGF shRNA demonstrated herein to other NGF inhibitors without undue experimentation. Details of the pharmaceutical compositions and methods are presented in greater detail in this disclosure.

In a one aspect, a pharmaceutical composition for treating/preventing atrial fibrillation is provided. In some embodiments, the pharmaceutical composition includes a small hairpin RNA (shRNA) directed against a NGF gene (“NGF shRNA”). The shRNA can be a unimolecular RNA that includes a sense sequence, a loop region, and an antisense sequence (sometimes referred to as first and second regions), which together form a hairpin loop structure. Preferably, the antisense and sense sequences are substantially complementary to one other (about 80% complementary or more), where in certain embodiments the antisense and sense sequences are 100% complementary to each other. In certain embodiments, the antisense and sense sequences are too short to be processed by Dicer, and hence act through an alternative pathway to that of longer double-stranded RNAs (e.g., shRNAs having antisense and sense sequences of about 16 to about 22 nucleotides in length, e.g., between 18 and 19 nucleotides in length (e.g., an sshRNA). Additionally, the antisense and sense sequences within a unimolecular RNA of the invention can be the same length, or differ in length by less than about 9 bases. The loop can be any length, with the preferred length being from 0 to 4 nucleotides in length or an equivalent length of non-nucleotidic linker, and more preferably 2 nucleotides or an equivalent length of non-nucleotidic linker (e.g., a non-nucleotide loop having a length equivalent to 2 nucleotides). In one embodiment, the loop is: 5′-UU-3′ (rUrU) or 5′-tt-3′, where “t” represents deoxythymidine (dTdT). Within any shRNA hairpin, a plurality of the nucleotides are ribonucleotides. In the case of a loop of zero nucleotides, the antisense sequence is linked directly to the sense sequence, with part of one or both strands forming the loop. In a preferred embodiment of a zero-nt loop shRNA, the antisense sequence is about 18 or 19 nt and the sense sequence is shorter than the antisense sequence, so that one end of the antisense sequence forms the loop.

A hairpin of representative shRNA's can be organized in either a left-handed (L) hairpin (i.e., 5′-antisense-loop-sense-3′) or a right-handed (R) hairpin (i.e., 5′-sense-loop-antisense-3′). Furthermore, an shRNA may also contain overhangs at either the 5′ or 3′ end of either the sense sequence or the antisense sequence, depending upon the organization of the hairpin. Preferably, if there are any overhangs, they are on the 3′ end of the hairpin and comprise between 1 to 6 bases. The presence of an overhang is preferred for R-type hairpins, in which case a 2-nt overhang is preferred, and a UU or tt overhang is most preferred.

Modifications can be added to enhance shRNA stability, functionality, and/or specificity and to minimize immunostimulatory properties. For example, the overhangs can be unmodified, or can contain one or more specificity or stabilizing modifications, such as a halogen or O-alkyl modification of the 2′ position, or internucleotide modifications such as phosphorothioate modification. The overhangs can be ribonucleic acid, deoxyribonucleic acid, or a combination of ribonucleic acid and deoxyribonucleic acid.

In another non-limiting example of modifications that can be applied to left handed hairpins, 2′-O-methyl modifications (or other 2′ modifications, including but not limited to other 2′-O-alkyl modifications) can be added to nucleotides at position 15, 17, or 19 from the 5′ antisense terminus of the hairpin, or any two of those positions, or all three, as well as to the loop nucleotides and to every other nucleotide of the sense sequence except for nucleotides 9, 10 and 11 from the 5′-most nucleotide of the sense sequence (also called the 9.sup.th, 10.sup.th, and 11.sup.th nucleotides), which should have no modifications that block “slicing” activity. Any single modification or group of modifications described in the preceding sentence can be used alone or in combination with any other modification or group of modifications cited.

Ui-Tei, K. et al. (Nucl. Acids Res. (2008) 36 (22): 7100-7109; incorporated by reference in its entirety) observed that the specificity of siRNAs can be increased by modifying the seed region of one or both strands. Such modifications are applicable to shRNA's of the present disclosure. In another non-limiting example of modifications that can be applied to hairpins, nt 1-6 of the antisense sequence and nt 14-19 of the sense sequence can be 2′-O-methylated to reduce off-target effects. In a preferred embodiment, only nt 1-6 are modified from 2′-OH to 2′-H or 2′-O-alky.

As the sense sequence of an shRNA can potentially enter RISC and compete with the antisense (targeting) strand, modifications that prevent sense sequence phosphorylation are valuable in minimizing off-target signatures. Thus, desirable chemical modifications that prevent phosphorylation of the 5′ carbon of the 5′-most nucleotide of right-handed shRNA of the invention can include, but are not limited to, modifications that: (1) add a blocking group (e.g., a 5′-O-alkyl) to the 5′ carbon; or (2) remove the 5′-hydroxyl group (e.g., 5′-deoxy nucleotides) (see, e.g., WO 2005/078094; incorporated by reference in its entirety).

In addition to modifications that enhance specificity, modifications that enhance stability can also be added. In some embodiments, modifications comprising 2′-O-alkyl groups (or other 2′ modifications) can be added to one or more, and preferably all, pyrimidines (e.g., C and/or U nucleotides) of the sense sequence. Modifications such as 2′ F or 2′-O-alkyl of some or all of the Cs and Us of the sense sequence/region, respectively, or the loop structure, can enhance the stability of the shRNA molecules without appreciably altering target specific silencing. It should be noted that while these modifications enhance stability, it may be desirable to avoid the addition of these modification patterns to key positions in the hairpin in order to avoid disruption of RNAi (e.g., that interfere with “slicing” activity).

Additional stabilization modifications to the phosphate backbone may be included in the shRNAs in some embodiments of the present invention. For example, at least one phosphorothioate, phosphordithioate, and/or methylphosphonate may be substituted for the phosphate group at some or all 3′ positions of nucleotides in the shRNA backbone, or any particular subset of nucleotides (e.g., any or all pyrimidines in the sense sequence of the oligonucleotide backbone), as well as in any overhangs, and/or loop structures present. These modifications may be used independently or in combination with the other modifications disclosed herein.

Description of modified shRNAs of interest can be found in the following references, both of which are incorporated herein by reference in their entirety: Q. Ge, H. Eves, A. Dallas, P. Kumar, J. Shorenstein, S. A. Kazakov, and B. H. Johnston (2010) Minimal-length short hairpin RNAs: The Relationship of Structure and RNAi Activity. RNA 16(1): 106-17 (Epub Dec. 1, 2009); and Q. Ge, A. Dallas, H. Ilves, J. Shorenstein, M. A. Behlke, and B. H. Johnston (2010) Effects of Chemical Modification on the Potency, Serum Stability, and Immunostimulatory Properties of Short shRNAs. RNA 16(1):118-30 (Epub Nov. 30, 2009).

Modified shRNAs according to aspects of the present invention may include additional chemical modifications for any of a variety of purposes, including 3′ cap structures (e.g., an inverted deoxythymidine), detectable labels conjugated to one or more positions in the shRNA (e.g., fluorescent labels, mass labels, radioactive labels, etc.), or other conjugates that can enhance delivery, detection, function, specificity, or stability (e.g., amino acids, peptides, proteins, sugars, carbohydrates, lipids, polymers, nucleotides, polynucleotides, etc.). Combinations of additional chemical modifications may be employed as desired by the user.

Suitable NGF shRNAs include those nucleic acids ranging from about 20 nucleotides to about 80 nucleotides in length, wherein a portion of the nucleic acids have a double-stranded structural domain ranging from about 15 nucleotides to about 25 nucleotides in length. In some aspects, the shRNA can include modified bases or phosphodiester backbones to impart stability of the shRNA inside tissues and cells. An exemplary NGF shRNA comprises SEQ ID NO: 1. In some embodiments, any shRNAs capable of inhibiting NGF expression find use within the compositions and methods herein. In certain embodiments, NGF shRNA with 1, 2, 3, 4, 5, 6, 7, 8, 9, or 10 substitutions or deletions relative to SEQ ID NO: 1 are provided.

As is generally known in the art, commonly used oligonucleotides are oligomers or polymers of ribonucleic acid or deoxyribonucleic acid having a combination of naturally-occurring purine and pyrimidine bases, sugars and covalent linkages between nucleosides including a phosphate group in a phosphodiester linkage. However, it is noted that the term “oligonucleotides” also encompasses various non-naturally occurring mimetics and derivatives, i.e., modified forms, of naturally occurring oligonucleotides, as described herein.

shRNA molecules of the invention can be prepared by any method known in the art for the synthesis of DNA and RNA molecules. These include techniques for chemically synthesizing oligodeoxy-ribonucleotides and oligo-ribonucleotides well known in the art such as for example solid phase phosphoramidite chemical synthesis. Alternatively, RNA molecules can be generated by in vitro and in vivo transcription of DNA sequences encoding the antisense RNA molecule.

Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

shRNA molecules can be chemically synthesized using appropriately protected ribonucleoside phosphoramidites and a conventional DNA/RNA synthesizer. Custom shRNA synthesis services are available from commercial vendors such as Ambion (Austin, Tex., USA) and Dharmacon Research (Lafayette, Colo., USA).

Various well-known modifications to the DNA molecules can be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′O-methyl rather than phosphodiesterase linkages within the oligodeoxyribonucleotide backbone. An antisense nucleic acid of the invention can be constructed using chemical synthesis or enzymatic ligation reactions using procedures known in the art. An antisense oligonucleotide can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

The shRNA molecules herein can be various modified equivalents of the structures of any NGF shRNA. A “modified equivalent” means a modified form of a particular shRNA molecule having the same target-specificity (i.e., recognizing the same mRNA molecules that complement the unmodified particular shRNA molecule). Thus, a modified equivalent of an unmodified shRNA molecule can have modified ribonucleotides, that is, ribonucleotides that contain a modification in the chemical structure of an unmodified nucleotide base, sugar and/or phosphate (or phosphodiester linkage).

In some embodiments, modified shRNA molecules contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. Examples of the non-phosphorous containing backbones described above are known in the art, e.g., U.S. Pat. No. 5,677,439, each of which is herein incorporated by reference.

Modified forms of shRNA compounds can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxy methyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyl uridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyl adenosine, beta-D-mannosylqueosine, uridine-5-oxyacetic acid, 2-thiocytidine, threonine derivatives, and the like.

In addition, modified shRNA compounds can also have substituted or modified sugar moieties, e.g., 2′-O-methoxyethyl sugar moieties.

Preferably, the 3′ overhangs of the shRNAs of the present invention are modified to provide resistance to cellular nucleases. In one embodiment the 3′ overhangs comprise 2′-deoxyribonucleotides.

In some embodiments, provided herein are shRNA compounds targeted at different sites of the mRNA corresponding to NGF. Additionally, to assist in the design of shRNAs for the efficient RNA interference (RNAi)-mediated silencing of any target gene, several shRNA supply companies maintain web-based design tools that utilize these general guidelines for “picking” shRNAs when presented with the mRNA or coding DNA sequence of the target gene. Examples of such tools can be found at the web sites of Dharmacon, Inc. (Lafayette, Colo.), Ambion, Inc. (Austin, Tex.). As an example, picking shRNAs involves choosing a site/sequence unique to the target gene (i.e., sequences that share no significant homology with genes other than the one being targeted), so that other genes are not inadvertently targeted by the same shRNA designed for this particular target sequence.

Another criterion to be considered is whether or not the target sequence includes a known polymorphic site. If so, shRNAs designed to target one particular allele may not effectively target another allele, since single base mismatches between the target sequence and its complementary strand in a given shRNA can greatly reduce the effectiveness of RNAi-induced by that shRNA. Given that target sequence and such design tools and design criteria, an ordinarily skilled artisan apprised of the present disclosure should be able to design and synthesized additional sihRNA compounds useful in reducing the mRNA level of NGF.

In some embodiments, the present invention provides a composition of a polymer or excipient and one or more vectors encoding one or more shRNA molecules. The vector can be formulated into a pharmaceutical composition with suitable carriers and administered into a mammal using any suitable route of administration. Because of this precision, side effects typically associated with traditional drugs can be reduced or eliminated. In addition, shRNA are relatively stable, and like antisense, they can also be modified to achieve improved pharmaceutical characteristics, such as increased stability, deliverability, and ease of manufacture. Moreover, because shRNA molecules take advantage of a natural cellular pathway, i.e., RNA interference, they are highly efficient in destroying targeted mRNA molecules. As a result, it is relatively easy to achieve a therapeutically effective concentration of an shRNA compound in a subject.

shRNA compounds may be administered to mammals by various methods through different routes. They can also be delivered directly to a particular organ or tissue by any suitable localized administration methods such as direct injection into a target tissue. In some embodiments, shRNA compounds are electroporated into cells following their injection directly into the target tissue. Alternatively, they may be delivered encapsulated in liposomes, by iontophoresis, or by incorporation into other vehicles such as hydrogels, cyclodextrins, biodegradable nanocapsules, and bioadhesive microspheres.

In vivo inhibition of specific gene expression by RNAi injected intravenously has been achieved in various organisms including mammals. See, for example, Song E. et al. “RNA interference targeting Fas protects mice from fulminant hepatitis,” Nature Medicine, 9:347-351(2003); incorporated by reference in its entirety. One route of administration of shRNA molecules of the invention includes direct injection of the vector at a desired tissue site, such as for example, into diseased or non-diseased cardiac tissue, into fibrotic heart tissue, such as fibrotic PLA tissue. Generally, however, NGF shRNAs or expression vectors encoding NGF shRNAs are directly injected into myocardial tissue (e.g., atrial tissue) to effectively knock-down NGF protein expression, to inhibit nerve growth, and/or to reduce or altogether eliminate the presence of AF in a subject.

In some embodiments, one or more vectors comprising one or more of shRNA of the invention are readministered after a first administration at any time interval or intervals after the first administration.

In some embodiments, shRNA encoding nucleic acids are formulated in pharmaceutical compositions, which are prepared according to conventional pharmaceutical compounding techniques. See, e.g., Remington's Pharmaceutical Sciences, 18th Ed. (1990, Mack Publishing Co., Easton, Pa.). The pharmaceutical compositions of the invention comprise a therapeutically effective amount of the vector encoding shRNA. These compositions can comprise, in addition to the vector, a pharmaceutically acceptable excipient, carrier, buffer, stabilizer or other materials well known in the art. Such materials should be non-toxic and should not interfere with the efficacy of the active ingredient. The carrier can take a wide variety of forms depending on the form of preparation desired for administration, e.g., intravenous, oral, intramuscular, subcutaneous, intrathecal, epineural or parenteral.

When the vectors of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation

In some embodiments, vectors are suitably formulated and introduced into the environment of the cell by any means that allows for a sufficient portion of the sample to enter the cell to induce gene silencing, if it is to occur. Many formulations for vectors are known in the art and can be used so long as the vectors gain entry to the target cells so that it can act. For example, the vectors can be formulated in buffer solutions such as phosphate buffered saline solutions comprising liposomes, micellar structures, and capsids. The pharmaceutical formulations of the vectors of the invention can also take the form of an aqueous or anhydrous solution or dispersion, or alternatively the form of an emulsion or suspension. The pharmaceutical formulations of the vectors of the present invention may include, as optional ingredients, solubilizing or emulsifying agents, and salts of the type that are well-known in the art. Specific non-limiting examples of the carriers and/or diluents that are useful in the pharmaceutical formulations of the present invention include water and physiologically acceptable saline solutions. Other pharmaceutically acceptable carriers for preparing a composition for administration to an individual include, for example, solvents or vehicles such as glycols, glycerol, or injectable organic esters. A pharmaceutically acceptable carrier can contain physiologically acceptable compounds that act, for example, to stabilize or to increase the absorption of the shRNA encoding vector. Other physiologically acceptable carriers include, for example, carbohydrates, such as glucose, sucrose or dextrans, antioxidants, such as ascorbic acid or glutathione, chelating agents, low molecular weight proteins or other stabilizers or excipients, saline, dextrose solutions, fructose solutions, ethanol, or oils of animal, vegetative or synthetic origin. The carrier can also contain other ingredients, for example, preservatives.

It will be recognized that the choice of a pharmaceutically acceptable carrier, including a physiologically acceptable compound, depends, for example, on the route of administration of the composition. The composition containing the vectors can also contain a second reagent such as a diagnostic reagent, nutritional substance, toxin, or additional therapeutic agent. Many agents useful in the treatment of cardiac disease are known in the art and are envisioned for use in conjunction with the vectors of this invention.

Formulations of vectors with cationic lipids can be used to facilitate transfection of the vectors into cells. For example, cationic lipids, such as lipofectin, cationic glycerol derivatives, and polycationic molecules, such as polylysine, can be used. Suitable lipids include, for example, Oligofectamine and Lipofectamine (Life Technologies), which can be used according to the manufacturer's instructions.

In some embodiments, suitable amounts of vector are introduced and these amounts can be empirically determined using standard methods. Typically, effective concentrations of individual vector species in the environment of a cell will be about 50 nanomolar or less 10 nanomolar or less, or compositions in which concentrations of about 1 nanomolar or less can be used. In other aspects, the methods utilize a concentration of about 200 picomolar or less and even a concentration of about 50 picomolar or less can be used in many circumstances. One of skill in the art can determine the effective concentration for any particular mammalian subject using standard methods.

In some embodiments, the shRNA is administered in a therapeutically effective amount. The actual amount administered, and the rate and time-course of administration, will depend on the nature and severity of the condition, disease or disorder being treated. Prescription of treatment, for example, decisions on dosage, timing, etc., is within the responsibility of general practitioners or specialists, and typically takes account of the disorder, condition or disease to be treated, the condition of the individual mammalian subject, the site of delivery, the method of administration and other factors known to practitioners. Examples of techniques and protocols can be found in Remington's Pharmaceutical Sciences 18th Ed. (1990, Mack Publishing Co., Easton, Pa.).

Alternatively, targeting therapies can be used to deliver the shRNA encoding vectors more specifically to certain types of cell, by the use of targeting systems such as antibodies or cell specific ligands. Targeting can be desirable for a variety of reasons, e.g., if the agent is unacceptably toxic, or if it would otherwise require too high a dosage, or if it would not otherwise be able to enter the target cells.

In some embodiments, shRNA are delivered into mammalian cells, particularly human cells, by a gene therapy approach, using a DNA vector from which shRNA compounds in, e.g., small hairpin form (shRNA), can be transcribed directly. Recent studies have demonstrated that while double-stranded shRNAs are very effective at mediating RNAi, short, single-stranded, hairpin-shaped RNAs can also mediate RNAi, presumably because they fold into intramolecular duplexes that are processed into double-stranded shRNAs by cellular enzymes. This discovery has significant and far-reaching implications, since the production of such shRNAs can be readily achieved in vivo by transfecting cells or tissues with DNA vectors bearing short inverted repeats separated by a small number of (e.g., 3, 4, 5, 6, 7, 8, 9) nucleotides that direct the transcription of such small hairpin RNAs. Additionally, if mechanisms are included to direct the integration of the vector or a vector segment into the host-cell genome, or to ensure the stability of the transcription vector, the RNAi caused by the encoded shRNAs, can be made stable and heritable. Not only have such techniques been used to “knock down” the expression of specific genes in mammalian cells, but they have now been successfully employed to knock down the expression of exogenously expressed transgenes, as well as endogenous genes in the brain and liver of living mice.

Gene therapy is carried out according to generally accepted methods as are known in the art. See, for example, U.S. Pat. Nos. 5,837,492 and 5,800,998 and references cited therein; incorporated by reference in their entireties. Vectors in the context of gene therapy are meant to include those polynucleotide sequences containing sequences sufficient to express a polynucleotide encoded therein. If the polynucleotide encodes an shRNA, expression will produce the antisense polynucleotide sequence. Thus, in this context, expression does not require that a protein product be synthesized. In addition to the shRNA encoded in the vector, the vector also contains a promoter functional in eukaryotic cells. The shRNA sequence is under control of this promoter. Suitable eukaryotic promoters include those described elsewhere herein and as are known in the art. The expression vector may also include sequences, such as selectable markers, reporter genes and other regulatory sequences conventionally used.

Accordingly, the amount of shRNA generated in situ is regulated by controlling such factors as the nature of the promoter used to direct transcription of the nucleic acid sequence, (i.e., whether the promoter is constitutive or regulatable, strong or weak) and the number of copies of the nucleic acid sequence encoding a shRNA sequence that are in the cell. Exemplary promoters include those recognized by pol I, pol II and pol III. In some aspects, a preferred promoter is a pol III promoter, such as the U6 pol III promoter.

In some embodiments, provided herein are kits for inhibiting expression of a target gene in a cell, the kit including a chemically modified shRNA as described herein. A “kit” refers to any system for delivering materials or reagents for carrying out a method of the invention. In the context of reaction assays, such delivery systems include systems that allow for the storage, transport, or delivery of reaction reagents (e.g., chemically modified shRNA, culture medium, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. Such contents may be delivered to the intended recipient together or separately. For example, a first container may contain a chemically modified shRNA for use in an assay, while a second container contains culture media RNA delivery agents (e.g., transfection reagents).

As noted above, the subject kits can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control reagents, e.g., non-chemically modified shRNA.

The pharmaceutical compositions described herein have therapeutic efficacy in treating/preventing AF. A pharmaceutical composition comprising a NGF shRNA has demonstrable activity in an art-accepted canine model for human AF. The results of the NGF shRNA studies demonstrate the feasibility of a general strategy to inhibit NGF activity or expression using NGF inhibitors for the treatment/prevention of AF. Such inhibitor agents include oligonucleotide-based compounds that target the NGF mRNA or protein, such as RNAi molecules, antisense RNA, shRNAs, etc. directed against NGF mRNA and oligonucleotide-based aptamers directed against the NGF polypeptide. Furthermore, small molecule organic compounds, peptides, antibodies or other agents having anti-NGF activity by specifically binding to or otherwise interfering with NGF protein functionality also find use in the treatment and/prevention of AF. Embodiments described above for the administration, formulation, dosing, and use of NGF shRNA also find use with other agents for the inhibition of NGF activity or expression.

In some embodiments, NGF inhibitors comprise any suitable bioactive molecules (e.g., a molecule capable of inhibiting the function of NGF). In some embodiments, a MGF inhibitor comprises a macromolecule, polymer, a molecular complex, protein, peptide, polypeptide, nucleic acid, carbohydrate, small molecule, etc.

In some embodiments, an NGF inhibitor is an NGF inhibitory peptide. In some embodiments, the present invention provides peptides of any suitable amino acid sequence capable of inhibiting one or more alleles of NGF. In some embodiments, peptides provided by or encoded by the compositions of embodiments of the present invention may comprise any arrangement of any standard amino acids (e.g. alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine) or non-standard amino acids (e.g. D-amino acids, chemically or biologically produced derivatives of common amino acids, selenocysteine, pyrrolysine, lanthionine, 2-aminoisobutyric acid, dehydroalanine, etc.). In some embodiments, NGF inhibitory peptides are inhibitors to NGF.

In some embodiments, NGF inhibitory peptides are provided to a subject as isolated or purified peptides. In some embodiments, NGF inhibitory peptides are provided to a subject as nucleic acid molecules that encode such peptides. In some embodiments, peptides are optimized to enhance cell penetration (e.g., sequence optimization, sequence tag, tagged with a small molecule, etc.).

In some embodiments, an NGF inhibitor is provided from an isolated nucleic acid comprising a minigene, wherein said minigene encodes a modified NGF peptide, wherein the peptide blocks the site of interaction between NGF and NGF binding partners in a cell, such as a human cell. In addition, the minigene can further comprise one or more of a promoter, a ribosomal binding site, a translation initiation codon, and a translation termination codon.

In some embodiments, the NGF inhibitor is provided as an isolated or purified polypeptide.

In some embodiments, the present invention provides methods of inhibiting a NGF-mediated signaling event in a cell or tissue. These methods comprise administering to a cell or tissue, preferably a human cell or tissue, one of a modified NGF peptide and an isolated nucleic acid comprising a minigene which encodes a modified NGF peptide, whereby following the administration, the NGF peptide inhibits the NGF-mediated signaling event in the cell or tissue.

In some embodiments, an NGF inhibitor comprises a small molecule. In some embodiments, the present invention provides a small molecule inhibitor of NGF. In some embodiments, the present invention provides a small molecule drug or pharmaceutical compound configured to or capable of inhibiting NGF activity, function expression, or the like.

In some embodiments, the present invention provides RNAi molecules (e.g., that alter 5 NGF expression) as a NGF inhibitor. In some embodiments, the present invention targets the expression of NGF genes using nucleic acid based therapies. For example, in some embodiments, the present invention employs compositions comprising oligomeric antisense or RNAi compounds, particularly oligonucleotides, for use in modulating the function of nucleic acid molecules encoding NGF genes, ultimately modulating the amount of NGF protein expressed. In some embodiments, RNAi is utilized to inhibit NGF gene function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, an siRNA is an 18 to 30 nucleotide, preferably 19 to 25 nucleotide, most preferred 21 to 23 nucleotide or even more preferably 21 nucleotide-long double-stranded RNA molecule. siRNA is involved in the RNA interference (RNAi) pathway where the siRNA interferes with the expression of a specific gene (e.g., the NGF). siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest (e.g., the NGF).

Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, in some embodiments at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA and inhibit NGF is envisioned in the present invention. In some embodiments, siRNA duplexes are provided composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP). In some embodiments, provided herein are siRNA molecules that target and inhibit the expression (e.g., knock down) of NGF

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

Further molecules effecting RNAi (and useful herein for the inhibition of expression of NGF) include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of NGF after introduction into target cells. In some embodiments, provided herein are miRNA molecules that target and inhibit the expression (e.g., knock down) of NGF.

Morpholinos (or morpholino oligonucleotides) are synthetic nucleic acid molecules having a length of about 20 to 30 nucleotides and, typically about 25 nucleotides. Morpholinos bind to complementary sequences of target transcripts (e.g., NGF) by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knock down) of NGF.

A ribozyme (ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence can be created, provided the target sequence contains a potential matching cleavage site. The basic principle of constructing hammerhead ribozymes is as follows: A region of interest of the RNA (e.g., a portion of NGF), which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors of NGF.

In some embodiments, NGF expression is modulated using antisense compounds that specifically hybridize with one or more nucleic acids encoding NGF. The specific hybridization of an oligomeric compound with its target nucleic acid interferes with the normal function of the nucleic acid. This modulation of function of a target nucleic acid by compounds that specifically hybridize to it is generally referred to as “antisense.”

In some embodiments, the present invention contemplates the use of any genetic manipulation for use in modulating the expression of NGF genes. Examples of genetic manipulation include, but are not limited to, gene knockout (e.g., removing the NGF gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter.

In some embodiments, NGF expression is inhibited (and/or NGF activity is inhibited) by modifying the NGF sequence in target cells. In some embodiments, the alteration of NGF is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for the target sequence (e.g., a sequence within NGF) and fixed tracrRNA) are introduced into the cell. In general, target sites at the 5′ end of the gRNA target the Cas nuclease to the target site, e.g., NGF, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., sequence within NGF). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site, followed by disruptions or alterations as discussed herein. In other embodiments, Cas9 variants, deemed “nickases,” are used to nick a single strand at the target site (e.g., within NGF). Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression (e.g., to inhibit expression of NGF). In some embodiments, the CRISPR system is used to alter NGF, inhibit expression of NGF, and/or to inactivate the expression product of NGF. In some embodiments, using the CRISPR/Cas9 or a related system an NGF gene in a subject is altered in order to reduce the expression and/or activity of the NGF gene or resulting protein. In some embodiments, a nucleic acid encoding a NGF peptide or polypeptide, or an NGF inhibitor, is inserted into the genetic material of a host using a CRISPR/Cas9 system. CRISPRs are DNA loci comprising short repetitions of base sequences. Each repetition is followed by short segments of “spacer DNA” from previous exposures to a virus. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. The CRISPR/Cas system may be used for gene editing. By delivering the Cas9 protein and appropriate guide RNAs into a cell, the organism's genome can be cut at any desired location. Methods for using CRISPR/Cas9 systems, and other systems, for insertion of a gene into a host cell to produce an engineered cell are described in, for example, U.S. Pub. No. 20180049412; herein incorporated by reference in its entirety.

In some embodiments, the present invention provides antibodies that target NGF protein. Any suitable antibody (e.g., monoclonal, polyclonal, or synthetic) may be utilized in the therapeutic methods disclosed herein. In preferred embodiments, the antibodies are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the present invention provides methods of enhancing entry of an NGF inhibitor into cells or tissue. In some embodiments, the present invention provides administering a NGF inhibitor in conjunction with electroporation, electropermeabilization, or sonoporation. In some embodiments, the present invention provides administering a NGF inhibitor in conjunction with electroporation. In some embodiments, the present invention provides co-injection/electroporation of the tissue of a subject. In some embodiments, the present invention provides administering an NGF inhibitor prior to, simultaneously with, and/or following electroporation. In some embodiments, electroporation provides a method of delivering pharmaceuticals or nucleic acids (e.g. DNA) into cells. In some embodiments, tissue electrically stimulated at the same time or shortly after pharmaceutical or DNA is applied (e.g. NGF inhibitor). In some embodiments, electroporation increases cell permeability. The permeability or the pores are large enough to allow the pharmaceuticals and/or DNA to gain access to the cells. In some embodiments, the pores in the cell membrane close and the cell once again becomes impermeable or less permeable. Certain devices for co-injection/electroporation are known in the art (U.S. Pat. No. 7,328,064, herein incorporated by reference in its entirety).

The following patent applications contain compositions, devices, systems, and methods that may find use in embodiments herein: U.S. Pub. No. 20210038501; U.S. Pub. No. 20200237929; U.S. Pub. No. 20200206498; U.S. Pub. No. 20200185062; U.S. Pub. No. 20190111241; U.S. Pub. No. 20190076417; U.S. Pub. No. 20190032058; U.S. Pub. No. 20170172440; U.S. Pub. No. 20150366477; U.S. Pub. No. 20110137284; and U.S. Pub. No. 20090281019; each of which is incorporated by reference in their entireties.

Furthermore, though the canine model utilized PLA as model atrial tissue, the approach is applicable to all atrial tissues. In some embodiments, the present invention provides compositions and methods to treat or prevent atrial fibrillation. In some embodiments, the present invention provides treatment or prevention of a heart disease or condition selected from the list of aortic dissection, cardiac arrhythmia (e.g. atrial cardiac arrhythmia (e.g. premature atrial contractions, wandering atrial pacemaker, multifocal atrial tachycardia, atrial flutter, atrial fibrillation, etc.), junctional arrhythmias (e.g. supraventricular tachycardia, AV nodal reentrant tachycardia, paroxysmal supra-ventricular tachycardia, junctional rhythm, junctional tachycardia, premature junctional complex, etc.), atrio-ventricular arrhythmias, ventricular arrhythmias (e.g. premature ventricular contractions, accelerated idioventricular rhythm, monomorphic ventricular tachycardia, polymorphic ventricular tachycardia, ventricular fibrillation, etc.), etc.), congenital heart disease, myocardial infarction, dilated cardiomyopathy, hypertrophic cardiomyopathy, aortic regurgitation, aortic stenosis, mitral regurgitation, mitral stenosis, Ellis-van Creveld syndrome, familial hypertrophic cardiomyopathy, Holt-Orams Syndrome, Marfan Syndrome, Ward-Romano Syndrome, and/or similar diseases and conditions.

### EXPERIMENTAL

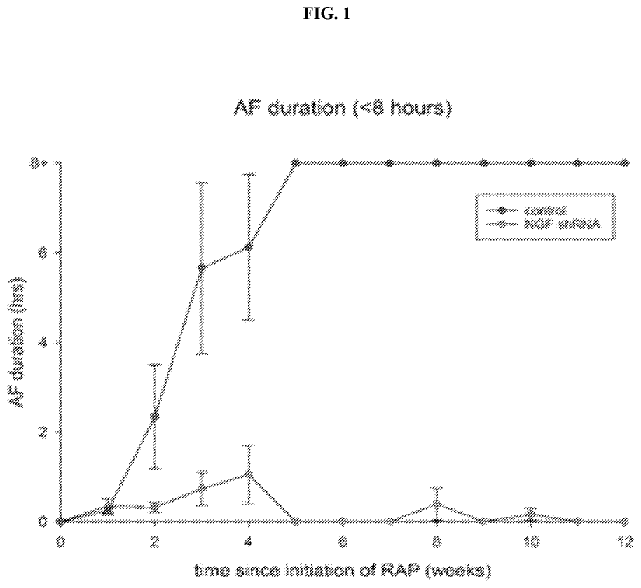

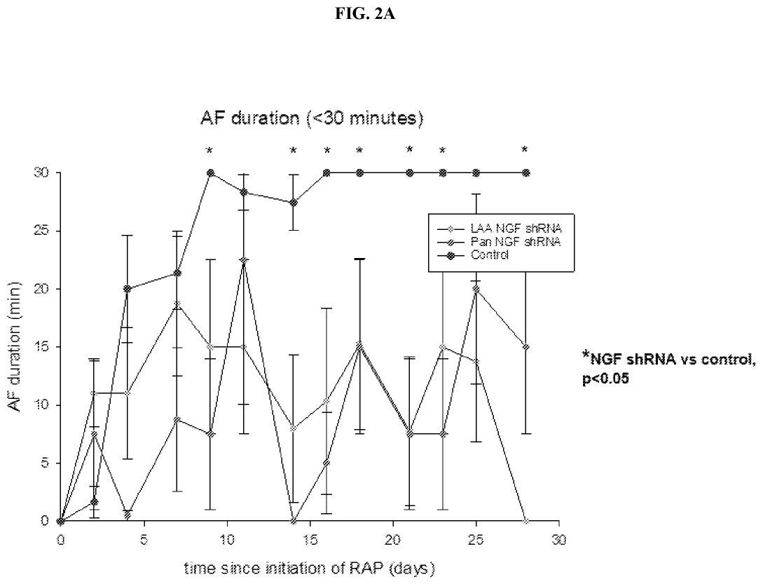

Canines subjected to more than a few weeks of rapid atrial pacing (RAP) exhibit an increase in nerve growth factor (NGF) secretion from the left atrial appendage (LAA), which is contemplated to be due to AF being more regular/organized in this region, thereby leading to more regular myocyte activation (Ref. 30; incorporated by reference in its entirety). Since an increasing number of studies indicate an important role for the LAA in development of persistent AF, it was contemplated that preferential NGF secretion in the LAA, via retrograde transport to atrial ganglionated plexi and stellate ganglia leads to diffuse autonomic nerve sprouting in the atria. In experiments conducted during development of embodiments herein, targeted injection of NGF shRNA (of SEQ ID NO: 1 or 2) in the LAA of canine subjects, followed by 4 weeks of RAP, resulted in a dramatic reduction of AF duration. Unlike control animals, no dog receiving NGF shRNA developed AF during follow up (FIG. 1).

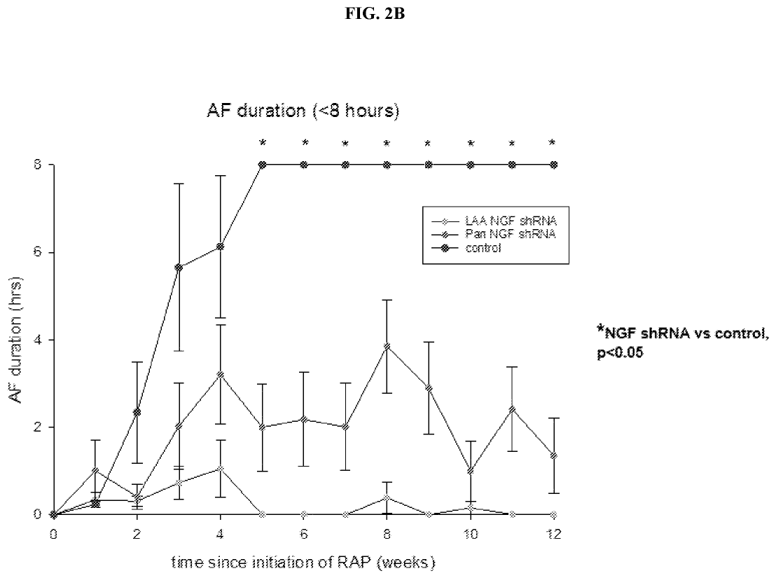

Extended experiments follow animals for periods of up to 12 weeks following 12 weeks-12 months) after by injection of the NGF shRNA in both the left and right atria similarly produced suppressed AF duration over time periods of 28 days (FIG. 2A) and 12 weeks (FIG. 2B).

It is contemplated that detailed assessment of neural innervation in both atria following targeted gene injection will reveal that targeted inhibition of NGF in the LAA prevents RAP induced nerve sprouting and thereby prevent progression of paroxysmal to persistent AF.

SEQUENCES

SEQ ID NO: 1-CACTGGACTAAACTTCAGCAT
SEQ ID NO: 2-GCATAGCGTAATGTCCATGTT

1. A method of treating and/or preventing atrial fibrillation (AF) in a subject, comprising administering an effective amount of a nerve growth factor (NGF) inhibitory agent to the subject.

2. The method of claim 1, wherein the subject suffers from atrial fibrillation.

3. The method of claim 1, wherein the NGF inhibitory agent inhibits the expression of NGF.

4. The method of claim 1, wherein the NGF inhibitory agent comprises a nucleic acid.

5. The method of claim 4, wherein administering the nucleic acid comprises administering a vector and/or transgene encoding the nucleic acid and allowing the nucleic acid to be expressed within the cells of the subject.

6. The method of claim 4, wherein administering the nucleic acid comprises directly administering the nucleic acid to the subject.

7. The method of claim 4, wherein the nucleic acid is an antisense RNA, short hairpin RNA (shRNA), short interfering RNA (siRNA), or microRNA (miRNA).

8. The method of claim 4, wherein the nucleic acid is an NGF shRNA comprising 70% sequence identity with SEQ ID NO: 1.

9. The method of claim 8, wherein the NGF shRNA comprising 100% sequence identity with SEQ ID NO: 1.

10. The method of claim 1, wherein the NGF inhibitory agent is administered to the myocardial tissue of the subject.

11. The method of claim 10, wherein the myocardial tissue comprises atrial or ventricle tissue.

12. The method of claim 11, wherein the NGF inhibitory agent is administered to the left and/or right atrial tissue.

13. The method of claim 12, wherein the NGF inhibitory agent is administered to the left atrial appendage.

14. The method of claim 1, wherein administering the NGF inhibitory agent comprises injecting the NGF inhibitory agent into the tissue of the subject.

15. The method of claim 14, wherein the injecting is by needleless injection.

16. The method of claim 1, further comprising assessing a parameter of atrial tissue status in the subject.

17. The method of claim 16, wherein assessing a parameter of atrial tissue status in the subject comprises monitoring an electrophysiological measurement associated with AF or assessing nerve sprouting for a region of the myocardial tissue before and/or after administering the NGF inhibitory agent to the subject.

18. The method of claim 17, wherein assessing a parameter of atrial tissue status in the subject comprises monitoring an electrophysiological measurement associated with AF selected from AF onset, AF duration, AF episode inducibility, effective refractory periods, conductivity, and conductive inhomogeneity index.

19. A composition comprising a nucleic acid capable of inhibiting expression of nerve growth factor (NGF).

20. The composition of claim 19, wherein the nucleic acid is an antisense RNA, short hairpin RNA (shRNA), short interfering RNA (siRNA), or microRNA (miRNA).

21. The composition of claim 19, wherein the nucleic acid is a vector or transgene encoding an antisense RNA, short hairpin RNA (shRNA), short interfering RNA (siRNA), or microRNA (miRNA).

22. The composition of claim 19, wherein the nucleic acid is an isolated nucleic acid encoding a small hairpin RNA against NGF mRNA.

23. The composition of claim 22, wherein the nucleic acid is an NGF shRNA comprising 70% sequence identity with SEQ ID NO: 1.

24. The composition of claim 23, wherein the NGF shRNA comprising 100% sequence identity with SEQ ID NO: 1.
